(19) United States
(12) Patent Application Publication
(10) Pub. No.: US 2017/0056152 A1
(43) Pub. Date: Mar. 2, 2017

(54) Pre-Curved Stent Graft and Methods for Use

(71) Applicant: Sanford Health, Sioux Falls, SD (US)

(72) Inventor: Patrick W. Kelly, Sioux Falls, SD (US)

(21) Appl. No.: 15/253,303

(22) Filed: Aug. 31, 2016

Related U.S. Application Data
(60) Provisional application No. 62/212,562, filed on Aug. 31, 2015.

(51) Int. Cl. A61F 2/07; A61F 2/95; A61F 2/844

(52) U.S. Cl. CPC A61F 2/07; A61F 2/844; A61F 2/95; A61F 2002/826

(57) ABSTRACT

The present disclosure provides a stent graft having a lumen, the stent graft comprising (a) a first side and a second side opposite the first side, wherein the first side and the second side of the stent graft extend between a first end and a second end of the stent graft, and (b) a longitudinal support extending between the first end and the second end of the stent graft and coupled to one of the first side or the second side of the stent graft, and wherein, in an expanded condition, the stent graft has a radius of curvature and has a length of the first side that is longer than a length of the second side.

### RELATED APPLICATIONS

This application claims priority to U.S. Provisional Application No. 62/212,562 entitled “Pre-Curved Main Body Stent graft,” filed on Aug. 31, 2015, which is hereby incorporated by reference in its entirety.

### BACKGROUND THE INVENTION

Aortic aneurysms are a bulge in the aorta caused by weakening of the aortic wall and increased blood pressure. Over time, an aortic aneurysm can grow to a point where it may rupture resulting in patient death. Traditionally, such aneurysms are treated using open surgical techniques which require extensive surgical exposure, aortic clamping, and prolonged ischemic times. More recently, endovascular aneurysm repair has been introduced as an alternative. Endovascular aneurysm repair involves achieving remote access using wires and catheters. The stent graft used in such a procedure may have a nitinol self-expanding stent which provides structural characteristics for the device outside of the catheter. The stent graft may also include a fluid tight graft material attached to the self-expanding stent. The ends of the stent graft are deployed within a healthy segment of aorta or artery with a diameter oversized by approximately 20%. Oversizing the device diameter creates a seal which prevents blood from flowing into the aneurysmal sac. When all inlets and outlets to the aneurysm are sealed, the aneurysm is considered to be excluded, thereby eliminating blood pressure from the sac wall effectively reducing the risk of aneurysm rupture. However, if any of the points of seal are compromised, the aneurysmal sac can be repressurized, effectively reintroducing the risk of rupture. This less invasive technique has achieved positive outcomes in the last decade in the abdominal aorta and thoracic aorta.

More recently, endovascular repair has been used in the branched regions of the visceral aorta and aortic arch, leading physicians to treat aortic aneurysms with endovascular techniques more aggressively. These aggressive approaches challenge the stent graft's ability to achieve adequate seal with the vessel wall. The “bird beak” is a well characterized phenomenon that is common when stent grafts are used to repair aortic aneurysms that are near the aortic arch. The curvature at the distal arch or proximal descending thoracic aorta is significant. If a stent graft originates or terminates at that curvature, there will be poor seal along the lower curvature due to inadequate conformability. The incomplete seal can lead to eventual endoleak, failure of the system, and possible aneurysm rupture.

### SUMMARY OF THE INVENTION

The stent graft of the present invention may be used to exclude an ascending transverse and/or proximal descending aortic aneurysms, for example. Aneurysms that occur in this curved segment of the aorta may present issues with seal and fixation due to oblique deployment of the stent graft, as well as challenges in determining an appropriate diameter and length of the stent graft due to the radius of curvature of the native vessel. The stent graft disclosed herein beneficially provides a stent graft having a predetermined radius of curvature in an expanded condition. The stent graft also advantageously provides a longer length on one side of the lumen that corresponds to the side having a larger radius of curvature in the expanded condition. Both the pre-curved nature of the stent graft and different lengths of opposing sides of the lumen may increase the amount and quality of direct aortic wall contact and thus improve seal and fixation.

Thus, a first aspect of the disclosure provides a stent graft having a lumen, the stent graft includes (a) a first side and a second side opposite the first side, wherein the first side and the second side of the stent graft extend between a first end and a second end of the stent graft, and (b) a longitudinal support extending between the first end and the second end of the stent graft and coupled to one of the first side or the second side of the stent graft, and wherein, in an expanded condition, the stent graft has a radius of curvature and a length of the first side is longer than a length of the second side.

A second aspect provides a stent graft having a lumen, the stent graft includes a graft material coupled to a radial stent structure. The stent graft has a first side and a second side opposite the first side. The first side and the second side of the stent graft extend between a first end and a second end of the stent graft. The radial stent structure includes a shape memory alloy. In an expanded condition, the radial stent structure has a first radius of curvature for a portion of the radial stent structure arranged along the second side of the stent graft and has a second radius of curvature for a portion of the radial stent structure arranged along the first side of the stent graft. And the first side of the stent graft has a length that is longer than a length of the second side of the stent graft.

A third aspect provides a method for placement of a stent graft that includes: (a) introducing a guidewire into an arterial configuration via arterial access, (b) loading a delivery catheter containing the stent graft of the first aspect or second aspect onto the guidewire, (c) moving the delivery catheter along the guidewire and introducing the delivery catheter into the arterial configuration via arterial access, and (d) deploying the stent graft into at least one of the arterial configuration and a lumen of a previously-placed stent graft.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A is a side perspective view of a stent graft, according to an example embodiment, having one example longitudinal support.

FIG. 1B is a side perspective view of the stent graft, according to an example embodiment, having another example longitudinal support.

FIG. 2 is a side perspective view the stent graft, according to an example embodiment, having another example longitudinal support.

FIG. 3A is a bottom view of a stent graft in a compressed position, according to an example embodiment, having a further example longitudinal support.

FIG. 3B a side perspective view the stent graft of FIG. 3A in an expanded position.

FIG. 4 is a side perspective view of a stent graft, according to an example embodiment, having a longitudinal support disposed along a first portion of the stent graft.

FIG. 5 is a side perspective view of a stent graft, according to an example embodiment, having radial stents.

FIG. 6 is a side perspective view of a stent graft, according to one example embodiment, having a bare metal stent coupled to one end of the stent graft.

FIG. 7 is a side perspective view of a stent graft, according to one example embodiment, having a bare metal stent coupled to one end of the stent graft.

FIG. 8 is a side perspective view of a stent graft, according to one example embodiment.

### DETAILED DESCRIPTION OF THE INVENTION

Exemplary devices and methods are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, with respect to measurements, “about” means +/−5%.

As used herein, diameter ranges pertain to an unconstrained, ex vivo state of the stent graft and stent graft extensions. When the stent graft and stent graft extensions are in a deployed, in vivo state the diameter ranges will be on the order of about 10-20% smaller in diameter than the ex vivo state.

As used herein, “first end” refers to the end of the stent graft that will be a “proximal end” upon deployment in vivo through which blood flow enters the lumen of the stent graft.

As used herein, “second end” refers to the end of the stent graft that will be a “distal end” upon deployment in vivo through which blood flow exits the lumen of the stent graft. The “second end” is the end of the stent graft relative to the delivery catheter, such that the second end is the end that is initially unsheathed during deployment.

As used herein, a “stent graft” is a tubular, radially-expandable device comprising a fabric supported by a stent, and may be used to bridge aneurysmal arteries. As such, the term stent graft may be used herein to include bridging stent grafts. Such stent grafts and methods for their deployment and use are known to those of skill in the art. For example, vascular sheaths can be introduced into the patient's arteries, through which items, including but not limited to, guidewires, catheters and, eventually, the stent graft, may be passed.

As used herein, a “stent” is typically a cylindrical frame and means any device or structure that adds rigidity, expansion force, or support to a prosthesis, while “stent graft” refers to a prosthesis comprising a stent and a graft material associated therewith that forms a lumen through at least a portion of the length of the stent. A “graft” is a cylindrical liner that may be disposed on the stent's interior, exterior or both. A wide variety of attachment mechanisms are available to join the stent and graft together, including but not limited to, sutures, adhesive bonding, heat welding, and ultrasonic welding.

The stent can be made of any suitable material, including but not limited to biocompatible metals, implantable quality stainless steel wires, nickel and titanium alloys, and biocompatible plastics. The stents can either have material properties necessary to exhibit either self-expanding or balloon-expanding characteristics.

Any suitable graft material can be used. In a preferred embodiment, the graft material is a biocompatible fabric, including but not limited to woven or knitted polyester, such as poly(ethylene terephthalate), polylactide, polyglycolide and copolymers thereof; fluorinated polymers, such as PTFE, expanded PTFE and poly(vinylidene fluoride); polysiloxanes, including polydimethyl siloxane; and polyurethanes, including polyetherurethanes, polyurethane ureas, polyetherurethane ureas, polyurethanes containing carbonate linkages and polyurethanes containing siloxane segments. Materials that are not inherently biocompatible may be subjected to surface modifications in order to render the materials biocompatible. Examples of surface modifications include graft polymerization of biocompatible polymers from the material surface, coating of the surface with a cross-linked biocompatible polymer, chemical modification with biocompatible functional groups, and immobilization of a compatibilizing agent such as heparin or other substances. The graft material may also include extracellular matrix materials.

As used herein, a “catheter” is an apparatus that is connected to a deployment mechanism and houses a medical device that can be delivered over a guidewire. The catheter may include a guidewire lumen for over-the-wire guidance and may be used for delivering a stent graft to a target lumen. A catheter can have braided metal strands within the catheter wall to increase structural integrity. The structural elements of the catheter tip can be bonded or laser welded to the braided strands of the catheter to improve the performance characteristics of the catheter tip.

As used herein, a “guidewire” is an elongate structure used to guide endovascular devices through the vasculature that is made from such materials as nitinol, stainless steel, or various polymers, for example. Guidewires may be used for selecting target lumens and guiding catheters to target deployment locations. Guidewires are typically defined as wires used independently of other devices that do not come as part of an assembly.

As used herein, “lumen” refers to a passage within an arterial structure, such as the pulmonary arteries, stent grafts or the passage within the tubular housings or catheters through which the guidewire may be disposed.

As used herein, “radially outward” refers to a direction away from a longitudinal axis of a lumen of a stent graft.

As used herein, “radially inward” refers to a direction towards a longitudinal axis of a lumen of a stent graft.

As used herein, “longitudinal” refers to a direction along a longitudinal axis of a lumen of a stent graft.

As used herein, “tension bearing element” refers to an elongate structure made from such materials as nitinol, stainless steel, a GORE-TEX® Suture, a bio-compatible string, a cable, or various polymers, for example.

As used herein, an “aortic aneurysm” means a bulge in the aorta resulting from a weakening of the aortic wall and blood pressure that is at risk of rupturing.

As used herein, “bird beak” means a phenomenon of stent grafting in regions of aortic curvature where the stent graft is unable to conform to the curvature of the native vessel in which the stent graft is deployed, and a portion of the inner curve of the stent graft extends into the flow lumen of the native vessel, resulting in compromised seal.

As used herein, “seal” refers to the state of aneurysm repair in which the stent graft is deployed within healthy tissue of an inlet or outlet segment of the aorta or an artery with a roughly 20% oversized diameter of the stent graft such that blood flow through that inlet or outlet passes through the stent graft.

As used herein, “endoleak” refers to the state of an aneurysm repair in which the stent graft does not have adequate seal with the inlet or an outlet vessel to the aneurysmal sac and blood is allowed to flow past the stent graft and into the previously excluded aneurysmal sac effectively re-pressurizing the aneurysmal sac.

As used herein, “expanded condition” means a state of a stent graft when deployed in a target vessel.

As used herein, “radius of curvature” means the radius of a circle that touches a curve at a given point and has the same tangent and curvature at that point. The radius of curvature of a stent graft may refer to the radius of curvature of either side of the stent graft, or the radius of curvature of the longitudinal axis of the stent graft.

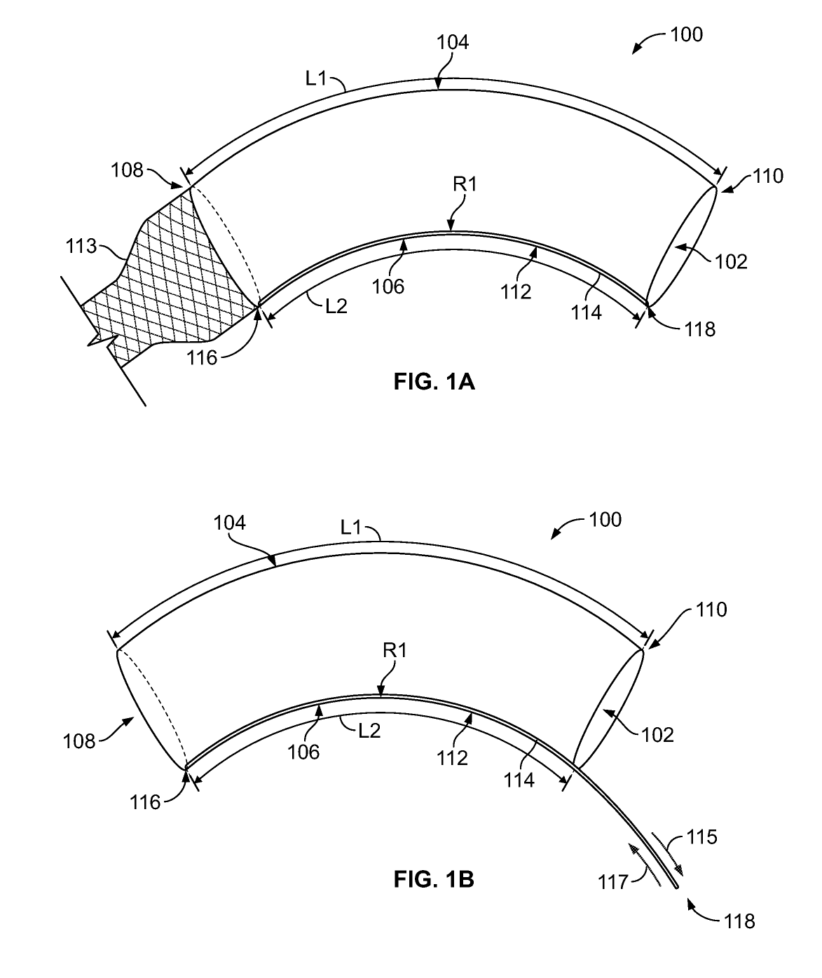

With reference to the Figures, FIG. 1A illustrates a stent graft 100 according to an example embodiment. The stent graft 100 defines a lumen 102. The stent graft 100 includes a first side 104 and a second side 106 opposite the first side 104. The first side 104 and the second side 106 of the stent graft 100 extend between a first end 108 and a second end 110 of the stent graft 100. The stent graft 100 further includes a longitudinal support 112 extending between the first end 108 and the second end 110 of the stent graft 100 and coupled to one of the first side 104 or the second side 106 of the stent graft 100. In an expanded condition, as shown in FIG. 1A, the stent graft 100 has a radius of curvature R1 and has a length L1 of the first side 104 that is longer than a length L2 of the second side 106. The radius of curvature R1 of the stent graft 100 may range from about 2 cm to about 200 cm. A diameter of the stent graft 100 may range from about 8 mm to about 65 mm. The length L1 of the first side 104 of the stent graft 100 in the expanded condition may range from about 10 mm to about 250 mm. The length L1 of the first side 104 is greater than the length L2 of the second side 106 by about 10 mm to about 100 mm.

The stent graft 100 may further include a stent valve 113 coupled to the firs end 108 of the stent graft 100. In this arrangement, a free end of the stent valve 113 may be covered and a portion of the stent valve 113 extending between the free end and the stent graft 100 may be uncovered. The stent valve 113 may be a percutaneous self-expanding valve affixed to the first end 108 of the stent graft 100 with the uncovered portion overlaying the coronary arteries to maintain blood flow. An exemplary embodiment of the stent valve 113 includes the Corevalve® manufactured by Medtronic. In one embodiment, the free end of the stent valve 113 may be covered with an impervious natural or synthetic material. In one embodiment, the stent valve 113 may be placed in the outflow tract of the aortic valve. The stent valve's anchoring mechanism is derived from, for example, a funnel shape with a larger diameter at the free end and smaller diameter at the point where the covered portion meets the uncovered portion.

In one example, as shown in FIG. 1A, the longitudinal support 112 may be a tension bearing element 114. As shown in FIG. 1A, the tension bearing element 114 may be coupled to the second side 106 of the stent graft 100. A first end 116 of the tension bearing element 114 may be coupled to the first end 104 of the stent graft 100. In one example, a length of the tension bearing element 114 may be shorter than the length L1 of the first side 104 of the stent graft 100. In such an example, a second end 118 of the tension bearing element may be coupled to the second end 108 of the stent graft 100, thereby imparting the radius of curvature R1 in the stent graft 100. Alternatively, the tension bearing element 114 may be coupled to the structure of the stent graft along the second side 106. In other embodiments, the tension bearing element 114 may be disposed within or coupled to the graft material of the stent graft along the second side 106. Such a tension bearing element 114 may comprise an elongate structure made from such materials as nitinol, stainless steel, a GORE-TEX® Suture, a bio-compatible string, a cable, or various polymers, for example. Further, such a tension bearing element 114 may be non-resistant to compression and resistant to tensile shear.

In another example, as shown in FIG. 1B, a length of the tension bearing element 114 may be greater than the length L1 of the first side 104 of the stent graft 100 such that the second end 118 of the tension bearing element 114 extends past the second end 110 of the stent graft 100. In such an example, the tension bearing element 114 may be slidably coupled to the second side 106 of the stent graft 100. For example, the tension bearing element 114 may be woven through the graft material of the stent graft 100. In such an embodiment, the length of the longitudinal support 112 may be adjustable to thereby adjust the radius of curvature R1 of the stent graft 100. In particular, the tension bearing element 114 may be moveable from a first position to a second position by moving the tension bearing element 114 in the direction 117. Further, the tension bearing element 114 may be moveable from the second position to the first position by moving the tension bearing element 114 in the direction 115. The second end 118 of the tension bearing element 114 is closer to the second end 110 of the stent graft 100 in the first position than in the second position. Further, the radius of curvature R1 of the stent graft 100 is greater in the first position than in the second position. As such, in operation a user may adjust the position of the tension bearing element 114 to achieve a desired radius of curvature R1 of the stent graft 100 to better fit a particular target arterial configuration of a particular patient.

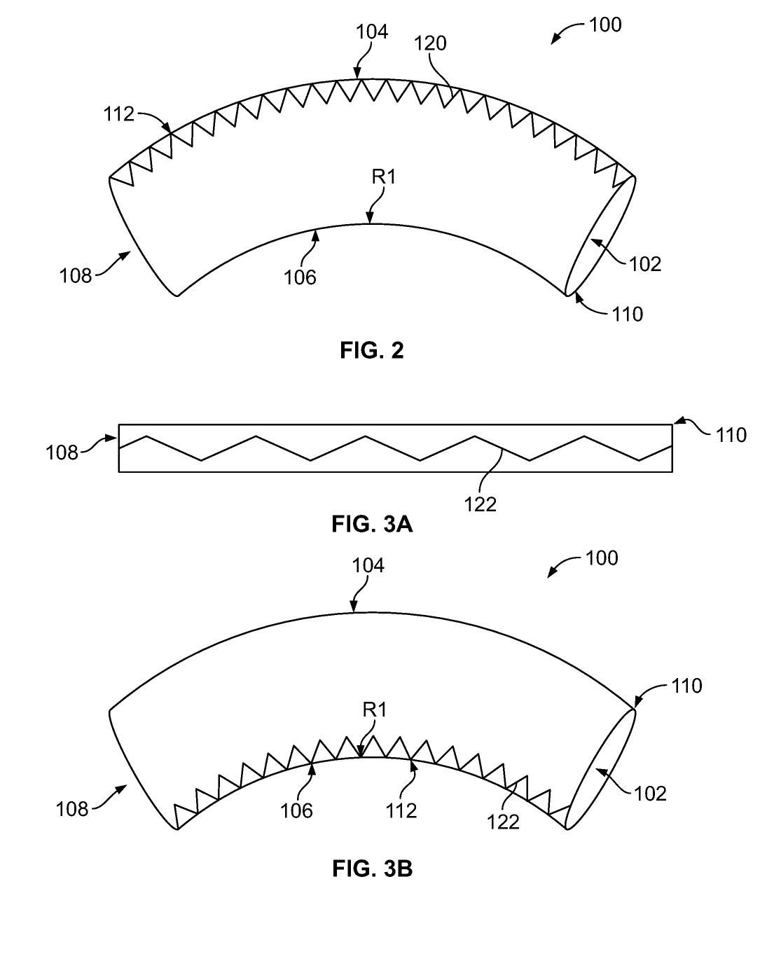

In another example, as shown in FIG. 2, the longitudinal support 112 may be a first stent 120 coupled to the first side 104 of the stent graft 100 and configured to expand longitudinally when the stent graft 100 is in the expanded condition. The first stent 120 may be made of a shape memory alloy, like nitinol, such that the first stent 120 is configured to be curved in the expanded condition imparting the radius of curvature R1 in the stent graft 100. As such, the first stent 120 may be biased to an expanded state when the stent graft 100 is in the expanded condition, thereby causing the length L1 of the first side 104 to be greater than the length L2 of the second side 106. The first stent 120 may have a zig-zag, sinusoidal or accordion shaped structure, as examples, which have a natural tendency to self-expand upon removal of a compressive force.

In another example, as shown in FIGS. 3A-3B, the longitudinal support 112 may include a second stent 122 coupled to the second side 106 of the stent graft 100. This second stent 122 may be standalone or used in conjunction with the first stent 121. The second stent 122 may be configured to contract longitudinally when the stent graft 100 is in the expanded condition. As shown in FIG. 3A, the stent graft 100 is in a compressed condition and the second stent 122 is stretched into or approaching a straightline. The second stent 122 may comprise a shape memory alloy, like nitionol, such that the second stent 122 is configured to be curved in the expanded condition imparting the radius of curvature R1 in the stent graft 100. In addition, the second stent 122 may be biased to a contracted state when the stent graft 100 is in the expanded condition as shown in FIG. 3B, thereby causing the length L2 of the second side 106 to be less than the length L1 of the first side 104. The second stent 122 may have a zig-zag, sinusoidal or accordion shaped structure, as examples.

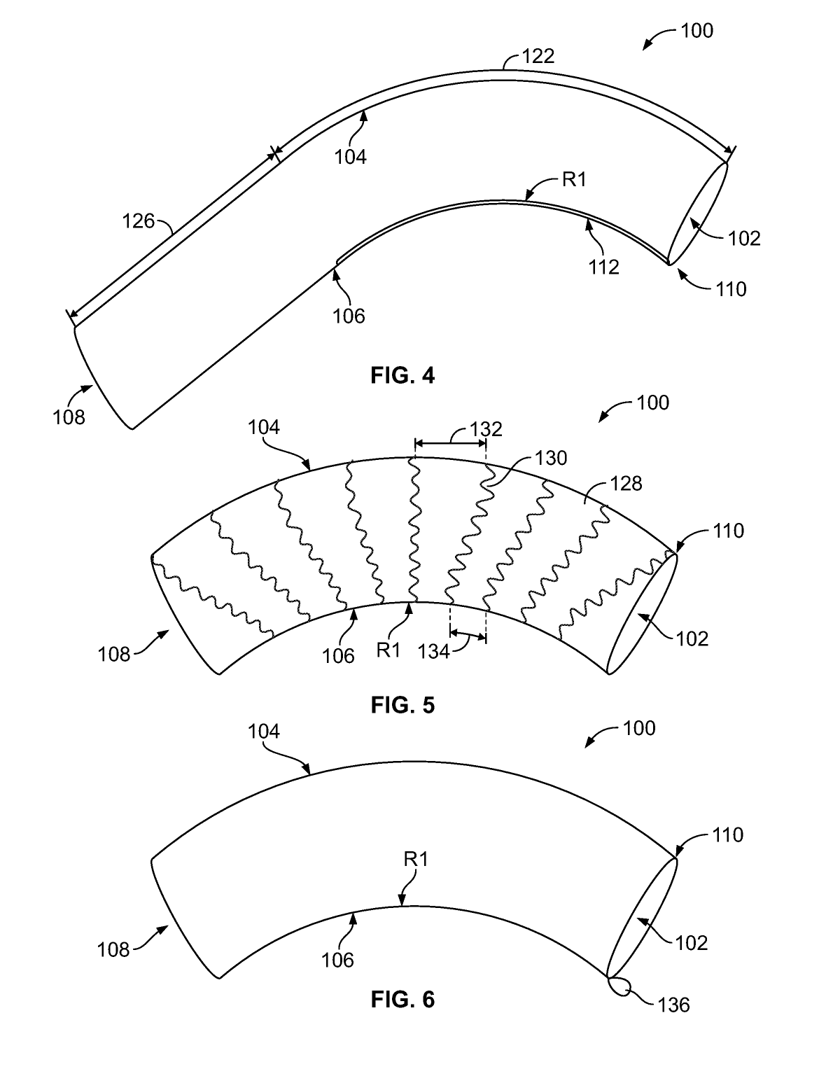

In one embodiment, as shown in FIG. 1A, the longitudinal support 112 may be coupled to the stent graft 100 such that the radius of curvature R1 extends along the entire length of the stent graft 100 in the expanded condition. In another embodiment, as shown in FIG. 4, the longitudinal support 112 may be coupled to a first portion 124 of the stent graft 100 such that the radius of curvature R1 extends along the first portion 124, while a second portion 126 of the stent graft 100 is substantially straight. In such an example, the first portion 124 may extend from a distal second end 110 of the stent graft 100 and the second portion 126 may be a proximal first end 108 of the stent graft 100, such that the distal end 110 of the stent graft 100 has a radius of curvature R1 while the proximal first end 108 of the stent graft 100 is substantially straight. Though the substantially straight first end 108, when deployed in vivo, may conform to the path of the lumen in which it resides. Such a configuration may be useful to better fit certain target arterial configurations.

Further, as shown in FIG. 5, graft material 128 of the stent graft 100 may be shorter on the second side 106 than graft material 128 on the first side 104 of the stent graft 100. In such an example, a plurality of radial stents 130 may be coupled to the graft material 128 along the stent graft 100 such that gaps 132 between the plurality of radial stents 130 along the first side 104 are greater in size than gaps 134 between the plurality of radial stents 130 along the second side 106, thereby imparting the radius of curvature R1 to the stent graft 100.

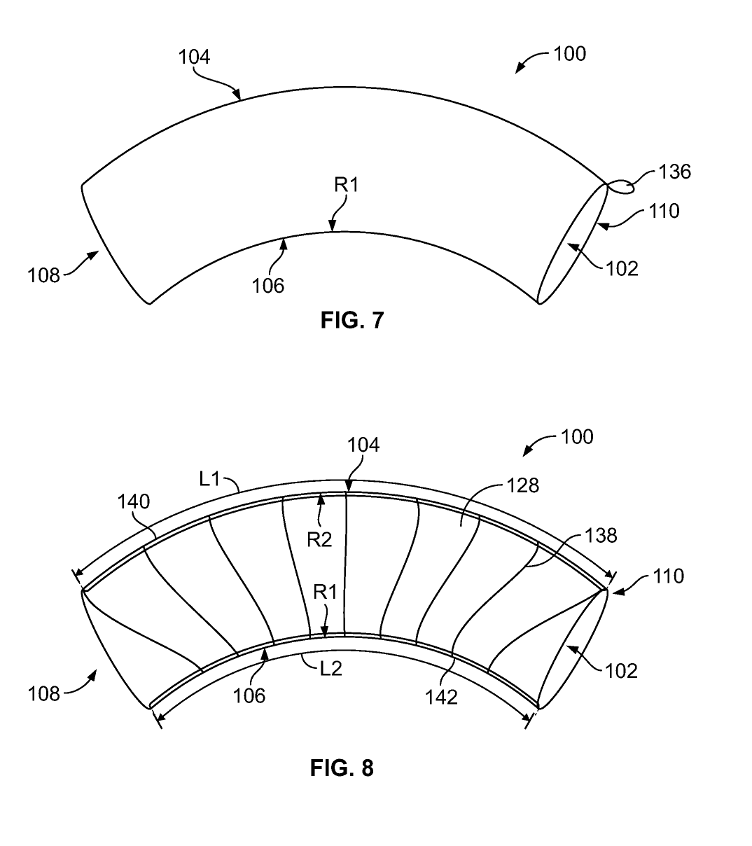

In yet another embodiment, as shown in FIGS. 6 and 7, a bare metal stent 136 may be coupled to the second end 110 of the stent graft 100 that is positioned closest to the tip of a deployment delivery system to aid in stent placement. Such a bare metal stent 136 may be coupled to the deployment system in an eccentric manner such that during deployment, there exists a bias of the stent graft 100 to one side of the native vessel into which the stent graft 100 is being deployed. In one embodiment, as shown in FIG. 6, the bare metal stent 136 is coupled to the second side 106 of the stent graft 100, and the length of the stent graft 100 in the compressed condition will correspond to the arc length of the native vessel that the stent graft 100 is intended to be deployed in. Such a configuration may aid in determining an appropriate size of the stent graft 100 on a patient-by-patient basis. In another embodiment, as shown in FIG. 7, the bare metal stent 136 is coupled to the first side 104 of the stent graft 100 and is thereby biased toward the vessel wall during deployment. In operation, such a configuration may advantageously permit more accurate placement and orthogonal deployment of the stent graft 100 in the native vessel.

In a further embodiment, as shown in FIG. 8, the stent graft 100 may comprise a graft material 128 coupled to a radial stent structure 138. In such an embodiment, the stent graft 100 has a first side 104 and a second side 106 opposite the first side 104. The first side 104 and the second side 106 of the stent graft 100 extend between a first end 108 and a second end 110 of the stent graft 100. The radial stent structure 138 includes a shape memory alloy, such as nitinol for example. In an expanded condition, the radial stent structure 138 has a first radius of curvature R1 for a portion of the radial stent structure 138 arranged along the second side 106 of the stent graft 100 and has a second radius of curvature R2 for a portion of the radial stent structure 138 arranged along the first side 104 of the stent graft 100. The first radius of curvature R1 is less than the second radius of curvature R2, such that the first side 104 of the stent graft 100 has a length that is longer than a length of the second side 106 of the stent graft 100.

In such an example, the stent graft 100 may further comprise a first longitudinal support 140 extending between the first end 108 and the second end 110 of the stent graft 100 and coupled to the first side 104 of the main body stent graft. In the expanded condition, the first longitudinal support 140 has a different radius of curvature than the first radius of curvature R1 of the radial stent structure 138. Such an arrangement may enable a user to adjust the radius of curvature R1 of the radial stent structure 138 in situ. The radius of curvature of the first longitudinal support 140 forces the stent graft 100 into a curved structure defined by the first longitudinal support, so that the stent graft 100 conforms to the shape of the curved structure.

In addition, such a stent graft 100 may include a second longitudinal support 142 extending between the first end 108 and the second end 110 of the stent graft 100 and coupled to the second side 106 of the stent graft 100. In the expanded condition, the second longitudinal support has a different radius of curvature than the second radius of curvature R2 of the radial stent structure 138. Such an arrangement may enable a user to adjust the radius of curvature R2 of the radial stent structure 138 in situ. The radius of curvature of the second longitudinal support 142 further forces the stent graft 100 into a curved structure defined by the first longitudinal support 140 and the second longitudinal support 142, so that the stent graft 100 conforms to the shape of the curved structure.

In operation, an example method for placement of a stent graft 100 may include (a) introducing a guidewire into an arterial configuration via arterial access, (b) loading a delivery catheter containing the stent graft 100 according to the embodiments described above onto the guidewire, (c) moving the delivery catheter along the guidewire and introducing the delivery catheter into the arterial configuration via arterial access, and (d) deploying the stent graft 100 into at least one of the arterial configuration and a lumen of a previously-placed stent graft. In one embodiment, the method may further include (e) coupling only the first side 104 of the stent graft 100 to a tip of the delivery catheter. In another example, the method may further include (f) adjusting a length of the longitudinal support 112, thereby adjusting the radius of curvature R1 of the stent graft 100.

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the invention can be combined unless the context clearly dictates otherwise. The various aspects and embodiments disclosed herein are for purposes of illustration and are not intended to be limiting, with the true scope and spirit being indicated by the claims.

1. A stent graft having a lumen, the stent graft comprising:
a first side and a second side opposite the first side, wherein the first side and the second side of the stent graft extend between a first end and a second end of the stent graft; and
a longitudinal support extending between the first end and the second end of the stent graft and coupled to one of the first side or the second side of the stent graft, and wherein, in an expanded condition, the stent graft has a radius of curvature and a length of the first side is longer than a length of the second side.

2. The stent graft of claim 1, wherein the longitudinal support comprises a tension bearing element, wherein the tension bearing element is coupled to the second side of the stent graft, and wherein a first end of the tension bearing element is coupled to the first end of the stent graft.

3. The stent graft of claim 2, wherein a length of the tension bearing element is shorter than the length of the first side of the stent graft, and wherein a second end of the tension bearing element is coupled to the second end of the stent graft imparting the radius of curvature in the stent graft.

4. The stent graft of claim 2, wherein a length of the tension bearing element is greater than the length of the first side of the stent graft such that a second end of the tension bearing element extends past the second end of the stent graft.

5. The stent graft of claim 4, wherein the tension bearing element is slidably coupled to the second side of the stent graft.

6. The stent graft of claim 5, wherein the tension bearing element is moveable from a first position to a second position, wherein the second end of the tension bearing element is closer to the second end of the stent graft in the first position than in the second position, and wherein the radius of curvature of the stent graft is greater in the first position than in the second position.

7. The stent graft of claim 1, wherein the longitudinal support comprises a first stent coupled to the first side of the stent graft and configured to expand longitudinally when the stent graft is in the expanded condition, wherein the first stent comprises a shape memory alloy that is configured to be curved in the expanded condition imparting the radius of curvature in the stent graft.

8. The stent graft of claim 7, wherein the first stent has a zig-zag, sinusoidal or accordion shaped structure.

9. The stent graft of claim 1, wherein the longitudinal support comprises a second stent coupled to the second side of the stent graft and configured to contract longitudinally when the stent graft is in the expanded condition, wherein the second stent comprises a shape memory alloy that is configured to be curved in the expanded condition imparting the radius of curvature in the stent graft.

10. The stent graft of claim 9, wherein the second stent has a zig-zag, sinusoidal or accordion-shaped structure.

11. The stent graft of claim 1, wherein the radius of curvature of the stent graft ranges from about 2 cm to about 200 cm.

12. The stent graft of claim 1, wherein a diameter of the stent graft ranges from about 8 mm to about 65 mm.

13. The stent graft of claim 1, wherein the length of the first side of the stent graft in the expanded condition ranges from about 10 mm to about 250 mm.

14. The stent graft of claim 1, wherein the length of the first side is greater than the length of the second side by about 10 mm to about 100 mm.

15. The stent graft of claim 1, wherein the longitudinal support is coupled to the stent graft such that the radius of curvature extends along the entire length of the stent graft in the expanded condition.

16. The stent graft of claim 1, wherein the longitudinal support is coupled to a first portion of the stent graft such that the radius of curvature extends along the first portion, and wherein a second portion of the stent graft is substantially straight.

17. The stent graft of claim 1, wherein a graft material of the stent graft is shorter on the second side than the graft material on the first side of the stent graft, and wherein a plurality of radial stents are coupled to the graft material along a length of the stent graft such that gaps between the plurality of radial stents along the first side are greater in size than gaps between the plurality of radial stents along the second side thereby imparting the radius of curvature to the stent graft.

18. The stent graft of claim 1, further comprising a bare metal stent coupled to the second end of the stent graft, wherein the bare metal stent is coupled to one of the first side or the second side of the stent graft.

19. The stent graft of claim 1, further comprising a stent valve coupled to the first end of the stent graft.

20. A stent graft having a lumen, the stent graft comprising:
a graft material coupled to a radial stent structure; and
a first side and a second side opposite the first side, wherein the first side and the second side of the stent graft extend between a first end and a second end of the stent graft, wherein the radial stent structure includes a shape memory alloy, wherein, in an expanded condition, the radial stent structure has a first radius of curvature for a portion of the radial stent structure arranged along the second side of the stent graft and has a second radius of curvature for a portion of the radial stent structure arranged along the first side of the stent graft, and wherein the first side of the stent graft has a length that is longer than a length of the second side of the stent graft.

21. The stent graft of claim 20, further comprising a first longitudinal support extending between the first end and the second end of the stent graft and coupled to the first side of the stent graft, wherein, in the expanded condition, the first longitudinal support has a different radius of curvature than the first radius of curvature of the radial stent structure.

22. The stent graft of claim 21, further comprising a second longitudinal support extending between the first end and the second end of the stent graft and coupled to the second side of the stent graft, wherein, in the expanded condition, the second longitudinal support has a different radius of curvature than the second radius of curvature of the radial stent structure.

23. A method for placement of a stent graft, the method comprising:
introducing a guidewire into an arterial configuration via arterial access;
loading a delivery catheter containing the stent graft according to claim 1 onto the guidewire;
moving the delivery catheter along the guidewire and introducing the delivery catheter into the arterial configuration via arterial access; and
deploying the stent graft into at least one of the arterial configuration and a lumen of a previously-placed stent graft.

24. The method of claim 23, further comprising:
coupling only the first side of the stent graft to a tip of the delivery catheter.

25. The method of claim 23, further comprising:
adjusting a length of the longitudinal support, thereby adjusting the radius of curvature of the stent graft.
